(19) United States
(12) Patent Application Publication
(10) Pub. No.: US 2023/0242579 A1
(43) Pub. Date: Aug. 3, 2023

(54) Methods for Improving Resolution of Heterodimeric Proteins from Impurities Using Affinity Chromatography

(71) Applicant: Regeneron Pharmaceuticals, Inc., Tarrytown, NY (US)

(72) Inventors: Cindy Chen, New York, NY (US); Andrew Barmasse, Mount Kisco, NY (US); Mark Chiboroski, Ossining, NY (US); Christopher Sevinsky, Watervliet, NY (US); Sean Dinn, Delmar, NY (US)

(21) Appl. No.: 18/095,839

(22) Filed: Jan. 11, 2023

Related U.S. Application Data
(60) Provisional application No. 63/298,745, filed on Jan. 12, 2022.
(60) Provisional application No. 63/430,477, filed on Dec. 6, 2022.

(51) Int. Cl. C07K 1/22; C07K 1/36; B01D 15/38

(52) U.S. Cl. CPC C07K 1/22; C07K 1/36; B01D 15/3809

(57) ABSTRACT

Methods of purifying a heterodimeric protein (e.g., a bispecific antibody) from impurities through a series of chromatographic cycles are disclosed. In various embodiments, the pH of the elution buffer is increased with increasing cycles within the series to maintain minimal contamination with a binding impurity, and without significant loss of recovery of the heterodimeric protein.

### CROSS-REFERENCE TO RELATED APPLICATIONS

This application claims the benefit under 35 USC § 119(e) of U.S. Provisional Application Nos.: 63/298,745, filed Jan. 12, 2022; and 63/430,477, filed Dec. 6, 2022, each of which is incorporated herein by reference in its entirety for all purposes.

### FIELD OF THE INVENTION

The present invention relates to improving the resolving longevity of an affinity chromatography column for the purification of protein products, e.g, the purification of heterodimeric proteins from a complex mixture of proteins. Specifically, the methods include performing a series of chromatographic cycles utilizing an increased elution pH at increasing cycles to minimize contamination with impurities while minimizing loss of recovery of the heterodimeric protein (e.g., a bispecific antibody).

### BACKGROUND

The purification of protein products often requires utilizing various chromatography steps to remove impurities such as host cell proteins, DNA and undesired species of the protein product.

Heterodimeric proteins, including multi- or bi-specific antibodies, can be formatted for purification using affinity chromatography. One such format is based upon a standard fully human IgG antibody having an improved pharmacokinetic profile and minimal immunogenicity (see U.S. Pat. No. 8,586,713, which is incorporated herein in its entirety). A single common light chain and two distinct heavy chains combine to form the bispecific antibody. One of the heavy chains contains a substituted Fc sequence (hereinafter “Fc*”) that reduces or eliminates binding of the Fc* to Protein A. For example, one such Fc* sequence contains H435R/Y436F (by EU numbering system; H95R/Y96F by IMGT exon numbering system) substitutions in the CH3 domain. Co-expression of the two heavy chains and the common light chain, results in three products: two of which are homodimeric for the heavy chains and one of which is the desired heterodimeric bispecific product. The Fc* sequence allows selective purification of the FcFc* bispecific product on commercially available affinity columns, due to intermediate binding affinity for Protein A compared to the high avidity FcFc heavy chain homodimer, or the weakly binding Fc*Fc* homodimer.

To achieve commercial scale purification of a heterodimeric protein (e.g., a bispecific antibody), good resolution between the FcFc homodimer, the Fc*Fc heterodimer, and the Fc*Fc* homodimer is required. However, repeated use of an affinity column over a number of cycles generally leads to an increase in contamination by the binding impurities, which may lead to batch failures. While such issues can be addressed by replacement of the column's resin (the protein-binding ligand affixed to a substrate), column replacement is expensive (˜$15K/L of resin), and introduces delays associated with the time for unpacking and repacking the column. For purification via affinity chromatography, the cost of producing a purified heterodimeric protein is a function of the number of cycles that can be performed with an affinity resin while maintaining acceptable purity and recovery rates. Thus, methods for improving column function over a greater number of cycles are desirable.

### BRIEF SUMMARY OF THE INVENTION

In one aspect of the disclosure, the present invention provides a method of purifying a heterodimeric protein, comprising: (a) performing a series of chromatographic cycles, wherein each cycle comprises: (i) introducing a mixture of a heterodimeric protein and impurities to an affinity matrix containing a protein-binding ligand, wherein the heterodimeric protein comprises first and second polypeptides with differing affinity for the protein-binding ligand, and wherein at least one impurity binds the protein-binding ligand and at least one impurity does not bind the protein-binding ligand; (ii) washing the affinity matrix with a first wash buffer at a first pH of from 5 to 9 to remove non-binding impurities; (iii) eluting the heterodimeric protein from the affinity matrix in a first elution buffer at a second pH; and (iv) washing the affinity matrix with a second wash buffer at a third pH of less than 4 to remove binding impurities; wherein the second pH is at a preliminary pH during a preliminary series of cycles within the series of chromatographic cycles, and the second pH is raised to a subsequent pH higher than the preliminary pH during a subsequent series of cycles within the series of chromatographic cycles, wherein the preliminary pH and the subsequent pH are within a range of from 4.0 to 5.2 and (b) collecting the heterodimeric protein from the affinity matrix in an eluate.

In some embodiments, the preliminary series of cycles consists of 20 cycles. In some embodiments, the preliminary series of cycles consists of 30 cycles. In some embodiments, the preliminary series of cycles consists of 40 cycles. In some embodiments, the preliminary series of cycles consists of 50 cycles. In some embodiments, the preliminary series of cycles consists of at least 50 cycles, at least 60 cycles, at least 70 cycles, or at least 80 cycles, or more.

In some embodiments, the subsequent series of cycles consists of at least 20 cycles. In some embodiments, the subsequent series of cycles consists of at least 50, at least 60, at least 70, or at least 80 cycles.

In some embodiments, the preliminary pH is from 4.0 to 4.2. In some cases, the preliminary pH is 4.1±0.05. In some embodiments, the subsequent pH is from 4.3 to 4.7. In some cases, the subsequent pH is 4.5±0.05.

In one aspect of the disclosure, the present invention provides a method of purifying a heterodimeric protein, comprising: (a) performing a series of chromatographic cycles, wherein each cycle comprises: (i) introducing a mixture of a heterodimeric protein and impurities to an affinity matrix containing a protein-binding ligand, wherein the heterodimeric protein comprises first and second polypeptides with differing affinity for the protein-binding ligand, and wherein at least one impurity binds the protein-binding ligand and at least one impurity does not bind the protein-binding ligand; (ii) washing the affinity matrix with a first wash buffer at a first pH of from 5 to 9 to remove non-binding impurities; (iii) eluting the heterodimeric protein from the affinity matrix in a first elution buffer at a second pH; and (iv) washing the affinity matrix with a second wash buffer at a third pH of less than 4 to remove binding impurities; (b) measuring a level of binding impurity in an eluate containing the heterodimeric protein following any one or more of the cycles within the series of chromatographic cycles, and comparing the measured level of binding impurity to a reference level of binding impurity, wherein if the measured level of binding impurity exceeds the reference level of binding impurity, then increasing the second pH in a subsequent cycle within the series of chromatographic cycles, wherein the second pH is within a range of from 4.0 to 5.2 during each cycle or subsequent cycle within the series of chromatographic cycles; and (c) collecting the heterodimeric protein from the affinity matrix in the eluate.

In some embodiments, the reference level of binding impurity is from 2% to 10%. In some cases, the reference level of binding impurity is from 3% to 7%. In some cases, the reference level of binding impurity is 5%±0.5%.

In some embodiments, the level of binding impurity in the eluate is measured following each cycle within the series of chromatographic cycles. In some embodiments, the level of binding impurity in the eluate is measured following every fifth cycle in the series of chromatographic cycles. In some embodiments, the level of binding impurity in the eluate is measured following every tenth cycle in the series of chromatographic cycles. In some embodiments, the level of binding impurity in the eluate is measured following a twentieth cycle in the series of chromatographic cycles. In some embodiments, the level of binding impurity in the eluate is measured following a fortieth cycle or a fiftieth cycle in the series of chromatographic cycles. In some cases, the eluate is collected over a series of cycles (e.g., five cycles, or ten cycles), and the level of binding impurity is measured in the combined eluate pool.

In some embodiments, the second pH is increased to a range of from 4.3 to 4.7 from a range of from 4.0 to 4.2 if the measured level of binding impurity exceeds the reference level of binding impurity. In some cases, the second pH is increased to 4.5±0.05 from 4.1±0.05 if the measured level of binding impurity exceeds the reference level of binding impurity.

In one aspect of the disclosure, the present invention provides a method of purifying a heterodimeric protein, comprising: (a) performing a series of chromatographic cycles, wherein each cycle comprises: (i) introducing a mixture of a heterodimeric protein and impurities to an affinity matrix containing a protein-binding ligand, wherein the heterodimeric protein comprises first and second polypeptides with differing affinity for the protein-binding ligand, and wherein at least one impurity binds the protein-binding ligand and at least one impurity does not bind the protein-binding ligand; (ii) washing the affinity matrix with a first wash buffer at a first pH of from 5 to 9 to remove non-binding impurities; (iii) eluting the heterodimeric protein from the affinity matrix in a first elution buffer at a second pH; and (iv) washing the affinity matrix with a second wash buffer at a third pH of less than 4 to remove binding impurities; wherein the second pH is at a primary pH during a primary series of cycles within the series of chromatographic cycles, the second pH is raised to a secondary pH higher than the primary pH during a secondary series of cycles that succeeds the primary series of cycles within the series of chromatographic cycles, and the second pH is raised to a tertiary pH higher than the secondary pH during a tertiary series of cycles that succeeds the secondary series of cycles within the series of chromatographic cycles, wherein the primary pH, the secondary pH, and the tertiary pH are within a range of from 4.0 to 5.2; and (b) collecting the heterodimeric protein from the affinity matrix in an eluate.

In some embodiments, the primary series of cycles comprises from 5 to 50 cycles. In some cases, the primary series of cycles comprises up to 20 cycles. In some cases, the primary series of cycles comprises up to 40 cycles.

In some embodiments, the secondary series of cycles comprises from 5 to 50 cycles. In some cases, the secondary series of cycles comprises from 10 to 25 cycles.

In some embodiments, the tertiary series of cycles comprises from 5 to 50 cycles. In some cases, the tertiary series of cycles comprises from 10 to 25 cycles.

In some embodiments, the primary pH is in a range of from 4.0 to 4.2. In some cases, the primary pH is 4.1±0.05. In some embodiments, the secondary pH is in a range of from 4.2 to 4.4. In some cases, the secondary pH is 4.3±0.05. In some embodiments, the tertiary pH is in a range of from 4.4 to 4.6. In some cases, the tertiary pH is 4.5±0.05.

In some embodiments, the second pH is raised to a 4th pH higher than the tertiary pH during a 4th series of cycles that succeeds the tertiary series of cycles within the series of chromatographic cycles, wherein the 4th pH is within a range of from 4.0 to 5.2.

In some embodiments, the second pH is raised to a 5th pH higher than the 4th pH during a 5th series of cycles that succeeds the 4th series of cycles within the series of chromatographic cycles, wherein the 5th pH is within a range of from 4.0 to 5.2.

In some embodiments, the second pH is raised to a 6th pH higher than the 5th pH during a 6th series of cycles that succeeds the 5th series of cycles within the series of chromatographic cycles, wherein the 6th pH is within a range of from 4.0 to 5.2.

In some cases, the secondary pH is a pH from 0.1 to 0.9 higher than the primary pH, the tertiary pH is a pH from 0.1 to 0.9 higher than the secondary pH, the 4th pH is a pH from 0.1 to 0.9 higher than the tertiary pH, the 5th pH is a pH from 0.1 to 0.9 higher than the 4th pH, and/or the 6th pH is a pH from 0.1 to 0.9 higher than the 5th pH, wherein the primary pH is in a range of from 4.0 to 4.2. In some embodiments, the primary pH is 4.1±0.05.

In some embodiments, each of the primary series of cycles, the secondary series of cycles, the tertiary series of cycles, the 4th series of cycles, the 5th series of cycles, and/or the 6th series of cycles comprises from 5 to 50 cycles within the series of chromatographic cycles.

In various embodiments of any of the methods discussed above or herein, the impurities comprise homodimeric species of the first and second polypeptides.

In various embodiments of any of the methods discussed above or herein, the protein-binding ligand is Protein A, and the affinity matrix comprises the Protein A ligand affixed to a substrate.

In some cases, the Protein A ligand is an engineered Protein A comprising a Z-domain tetramer, an engineered Protein A comprising a Y-domain tetramer, or an engineered Protein A that lacks D and E domains.

In some cases, the substrate is a particle and the affinity matrix comprises a multiplicity of the particles comprising a mean diameter of from 25 μm to 100 μm. In some embodiments, the particles comprise a mean diameter of from 40 μm to 60 um. In some embodiments, the particles comprise a mean diameter of from 45 μm to 55 um. In some embodiments, the particles comprise a mean diameter of about 50 μm.

In some cases, the substrate comprises any one or more of agarose, poly(styrene divinylbenzene), polymethacrylate, cellulose, controlled pore glass, and spherical silica.

In some cases, the particles comprise pores having a mean diameter of about 1100 Å.

In various embodiments of any of the methods discussed above or herein, the elution buffer comprises a salt at a concentration of at least 250 mM. In some cases, the salt concentration is greater than 300 mM or greater than 400 mM. In some cases, the salt concentration is about 500 mM.

In some embodiments, the salt is selected from a salt containing (i) Cl⁻, Br⁻, I⁻, NO₃⁻, N(CH₃)₄⁺, NH₄⁺, Cs⁺, Rb⁺, K⁺, Na⁺, H⁺, Ca²⁺, Mg²⁺, Al³⁺; (ii) combinations of Na⁺, H⁺, Ca²⁺, Mg²⁺ or Al³⁺ with Cl⁻, Br⁻, I⁻, NO₃⁻, or ClO₄⁻, or (iii) CaCl₂, MgCl₂ or NaCl.

In various embodiments of any of the methods discussed above or herein, the first polypeptide comprises a CH3 domain that is capable of binding to the protein-binding ligand and the second polypeptide comprises a CH3 domain that is not capable of binding to the protein-binding ligand.

In various embodiments of any of the methods discussed above or herein, in which the protein-binding ligand is Protein A, the first polypeptide comprises a CH3 domain that is capable of binding to Protein A and the second polypeptide comprises a CH3 domain that is not capable of binding to Protein A. In some cases, the second polypeptide comprises a H435R modification and a Y436F modification (EU numbering) in the CH3 domain.

In various embodiments of any of the methods discussed above or herein, the first pH is from 6 to 8.

In various embodiments of any of the methods discussed above or herein, the third pH is from 2.8 to 3.5.

In various embodiments of any of the methods discussed above or herein, the heterodimeric protein is an antibody. In various embodiments of the methods, the heterodimeric protein is a bispecific antigen-binding protein. In some embodiments, the bispecific antigen-binding protein is a bispecific antibody.

In various embodiments of any of the methods discussed above or herein, at least 85% of the heterodimeric protein is recovered in the eluate in each cycle within the series of chromatographic cycles. In some cases, at least 87% of the heterodimeric protein is recovered in the eluate in each cycle within the series of chromatographic cycles. In some cases, at least 89% of the heterodimeric protein is recovered in the eluate in each cycle within the series of chromatographic cycles.

In various embodiments of any of the methods discussed above or herein, the series of chromatographic cycles comprises 100 or more cycles.

In various embodiments of any of the methods discussed above or herein, the affinity matrix may be contacted with a basic solution having a pH of at least 11 following every cycle. In some cases, the affinity matrix is contacted with a basic solution having a pH of at least 11 following every three cycles. In some cases, the affinity matrix is contacted with a basic solution having a pH of at least 11 following every five cycles. In some cases, the affinity matrix is contacted with a basic solution having a pH of at least 11 following every seven cycles. In some embodiments, the pH of the basic solution is at least 12. In some embodiments, the basic solution comprises a base at a concentration of from 0.1 N to 0.5 N. In some cases, the base concentration is from 0.1 N to 0.3 N. In some embodiments, the basic solution comprises NaOH.

In various embodiments of any of the methods discussed above or herein, each cycle may further comprise (v) cleaning the affinity matrix by contacting the affinity matrix with a basic solution having a pH of at least 11. In some cases, the pH of the basic solution is at least 12. In some embodiments, the basic solution comprises a base at a concentration of from 0.1 N to 0.5 N. In some cases, the concentration is from 0.1 N to 0.3 N. In some embodiments, the basic solution comprises NaOH. In some embodiments, at least 75% of the heterodimeric protein is recovered in the eluate in each cycle within the series of chromatographic cycles, and the binding impurities do not exceed 6.5%. In some cases, at least 78% of the heterodimeric protein is recovered in the eluate in each cycle within the series of chromatographic cycles. In some cases, at least 80% of the heterodimeric protein is recovered in the eluate in each cycle within the series of chromatographic cycles.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

### BRIEF DESCRIPTION OF THE DRAWINGS

FIG. 1 is an illustration of an exemplary heterodimeric protein (e.g., Bispecific Fc*Fc) and associated impurities (homodimeric species) in accordance with an embodiment of the disclosure. The heterodimeric protein includes one polypeptide that binds a protein-binding ligand and one polypeptide that does not bind a protein-binding ligand (∅). The two illustrated impurities are homodimers, the non-binding impurity composed of two polypeptides that do not bind (∅) a protein-binding ligand, and the binding impurity composed of two polypeptides that bind a protein-binding ligand.

FIG. 2 is an illustration of an exemplary chromatographic cycle in accordance with an embodiment of the disclosure. As shown, the cycle includes loading a mixture of heterodimeric protein and impurities onto an affinity matrix, washing the affinity matrix to remove non-binding impurities, eluting the heterodimeric protein, and washing the affinity matrix to remove binding impurities. Binding of the binding impurity and the heterodimeric protein to the protein-binding ligand in the affinity matrix is illustrated in the first two panels.

FIG. 3 illustrates the relationship between elution pH and the presence of binding impurity in the eluate and the corresponding recovery rate for the heterodimeric protein (e.g., a bispecific antibody) in a naïve chromatography column.

FIGS. 4A and 4B illustrate the impact of increasing the elution pH in a naïve column (7 cycles) and a cycled column (84 cycles) on the presence of binding impurity in the eluate (FIG. 4A) and the corresponding recovery rate for the heterodimeric protein (e.g., a bispecific antibody) (FIG. 4B). The “Goal <5%” shown in FIG. 4A regarding binding impurity levels is exemplary, and may vary depending on the heterodimeric protein being purified.

FIGS. 5A and 5B illustrate design diagnostic parameters, including power analysis (FIG. 5A) and a fraction of design space plot (FIG. 5B). The power analysis determines the probability that the proposed design will be able to distinguish a parameter effect of a certain size. As shown in FIG. 5A, the power of the main effect terms are >0.7. As shown in FIG. 5B, the relative prediction variance is below 0.32 over 50% of the design space.

FIG. 6 illustrates a grayscale map of correlations evaluated in Example 3. As shown, all correlations are below 0.6, indicating a sufficiently orthogonal design. A table of the data corresponding to the map is also included in FIG. 6.

FIGS. 7A and 7B illustrate model prediction profilers of % bispecific yield (FIG. 7A) and % binding impurity (FIG. 7B). As resolve elution buffer pH decreases and hydroxide contact time increases, both bispecific yield and binding impurity levels increase. Additionally, as column loading increases, bispecific yield increases.

### DETAILED DESCRIPTION

Before the present invention is described, it is to be understood that this invention is not limited to particular methods and experimental conditions described, as such methods and conditions may vary. It is also to be understood that the terminology used herein is for describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### General

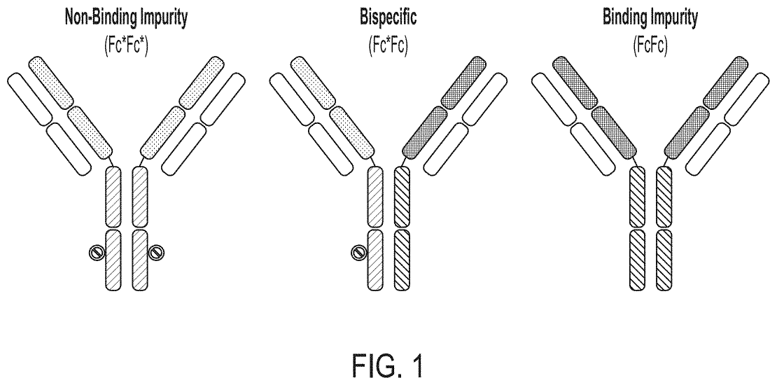

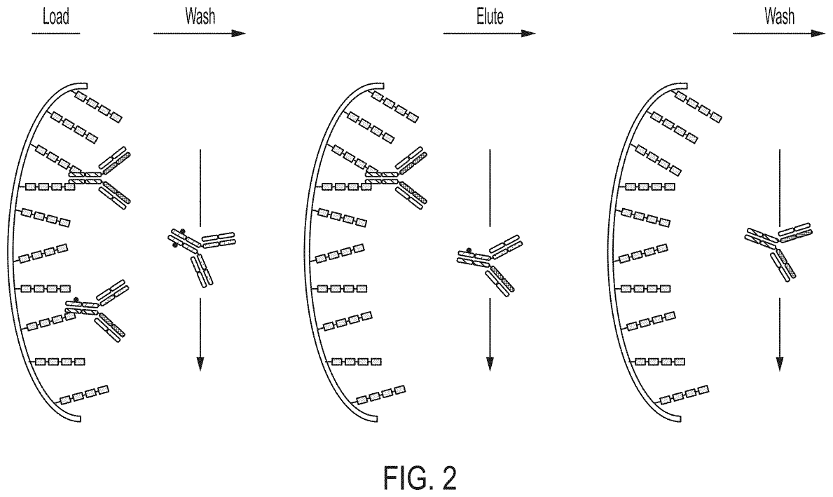

Purification of bispecific antibodies via affinity chromatography has been described previously. Briefly, a heterodimeric protein of interest that includes one polypeptide that binds a protein-binding ligand and one polypeptide that does not bind a protein-binding ligand, is introduced onto an affinity matrix (containing the protein-binding ligand) along with homodimeric impurities. As will be appreciated, the two homodimeric species include either a pair of polypeptides that binds the protein-binding ligand of the affinity matrix, or a pair of polypeptides that do not bind the protein-binding ligand of the affinity matrix (see, e.g., FIG. 1).

Repeated use of an affinity chromatography column over a number of cycles leads to a loss of functional protein-ligand density, resulting in an increase in impurity levels. Without intending to be bound by any particular theory, the loss of functional protein-ligand density is believed to result from a build-up of impurities, structural ligand changes, and/or a physical loss of ligand. In some cases, the loss of functional protein-ligand density is believed to be, at least partially, related to exposure to hydroxide ions (e.g., from NaOH) used to periodically clean the chromatography column. No matter the cause, the loss of functional protein-ligand density leads to lower avidity for the affinity matrix for both the binding impurities and the heterodimeric protein of interest. The lower avidity, coupled with a reduced probability for re-binding events, may lead to premature removal of the heterodimeric protein of interest with the non-binding impurity, or premature removal of the binding impurity with the heterodimeric protein of interest during elution.

The present invention is predicated, at least in part, on the unexpected discovery that increasing elution pH in a cycled affinity chromatography column can improve resolution of a heterodimeric protein (e.g., a bispecific antibody) from binding impurities while maintaining a high rate of recovery of the heterodimeric protein. Cost of materials for large-scale commercial manufacturing and purification of therapeutic proteins (e.g., bispecific antibodies) is a significant concern, wherein the cost of replacing a 100 L column can easily exceed $1.5 M, and delay purification processes. Thus, extending the usable lifetime of an affinity chromatography column over a greater number of cycles can achieve significant cost advantages.

As discussed in greater detail below, methods of prolonging affinity column resolution and maintaining heterodimeric protein recovery rates include: (i) performing a preliminary series of cycles at a preliminary elution pH and a subsequent series of cycles at a subsequent (and higher) pH; (ii) performing a series of cycles in which the impurity level in the eluate is measured after each cycle, or periodically, and raising the elution pH in a subsequent cycle or cycles to maintain a minimal impurity level throughout the series of cycles; and (iii) performing a series of cycles in which the elution pH is raised in a step-wise manner over multiple sets of cycles (e.g., the elution pH is raised from 0.1 to 1 point following every 5, 10, 15, 20, or 25 cycles). In addition, in some embodiments, reducing the cleaning frequency of the chromatography column (e.g., by contacting the column with a basic solution having a pH of at least 11), or reducing the concentration of the base in the solution used for cleaning the chromatography column can also prolong the affinity column resolution.

### Definitions

The term “antibody”, as used herein, includes immunoglobulin molecules comprised of four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10⁻⁹ M, at least 10⁻¹ M; at least 10⁻¹¹ M; or at least 10⁻¹² M, as measured by surface plasmon resonance, e.g., BIACORE™ or solution-affinity ELISA.

The phrase “bispecific antibody” includes an antibody capable of selectively binding two or more epitopes. Bispecific antibodies generally comprise two different heavy chains, with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

In various embodiments of the methods discussed herein, the heterodimeric proteins, bispecific antibodies, Fc-containing proteins, or the like, may be of isotype IgG. In some cases, the heterodimeric proteins, bispecific antibodies, Fc-containing proteins, or the like, are of isotype IgG1, IgG2, IgG3 or IgG4. In some cases, the heterodimeric proteins, bispecific antibodies, Fc-containing proteins, or the like are of isotype IgG1. In some cases, the heterodimeric proteins, bispecific antibodies, Fc-containing proteins, or the like, are of isotype IgG4. In various embodiments, the heterodimeric proteins, bispecific antibodies, Fc-containing proteins, or the like, are fully human.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (e.g., recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used with this invention include those, e.g., that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FR1, CDR1, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region,” or the term “CDR,” includes an amino acid sequence encoded by a nucleic acid sequence of an organism's immunoglobulin genes that normally (i.e., in a wild-type animal) appears between two framework regions in a variable region of a light or a heavy chain of an immunoglobulin molecule (e.g., an antibody or a T cell receptor). A CDR can be encoded by, for example, a germline sequence or a rearranged or unrearranged sequence, and, for example, by a naive or a mature B cell or a T cell. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as the result of splicing or connecting the sequences (e.g., V-D-J recombination to form a heavy chain CDR3).

The phrase “Fc-containing protein” includes antibodies, bispecific antibodies, heterodimeric proteins and immunoadhesins, and other binding proteins that comprise at least a functional portion of an immunoglobulin CH2 and CH3 region. A “functional portion” refers to a CH2 and CH3 region that can bind a Fc receptor (e.g., an FcγR; or an FcRn, i.e., a neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional.

Fc-containing proteins can comprise modifications in immunoglobulin domains, including where the modifications affect one or more effector function of the binding protein (e.g., modifications that affect FcγR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein and exhibits enhanced serum half-life (as compared with the same Fc-containing protein without the recited modification(s)) and have a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at 428 and/or 433 (e.g., L/R/SI/P/Q or K) and/or 434 (e.g., H/F or Y); or a modification at 250 and/or 428; or a modification at 307 or 308 (e.g., 308F, V308F), and 434. In another example, the modification can comprise a 428L (e.g., M428L) and 434S (e.g., N434S) modification; a 428L, 2591 (e.g., V2591), and a 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); a 307 and/or 308 modification (e.g., 308F or 308P).

The term “star substitution”, “Fc*”, and “HC*” includes any molecule, immunoglobulin heavy chain, Fc fragment, Fc-containing molecule, heterodimeric protein and the like which contain a sequence within the CH3 domain that abrogates binding to Protein A. Specific modifications, such as H95R and Y96F, that can diminish or abrogate Protein A binding in the CH3 domain are discussed in U.S. Pat. No. 8,586,713. This dipeptide mutation is designated as the “star substitution”.

The term “cell” includes any cell that is suitable for expressing a recombinant nucleic acid sequence. Cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell.

The phrase “mobile phase modifier” includes moieties that reduce the effect of, or disrupt, non-specific (i.e., non-affinity) ionic and other non-covalent interactions between proteins. “Mobile phase modifiers” include, for example, salts, ionic combinations of Group I and Group II metals with acetate, bicarbonate, carbonate, a halogen (e.g., chloride or fluoride), nitrate, phosphate, or sulfate. A non-limiting illustrative list of “mobile phase modifiers” includes beryllium, lithium, sodium, and potassium salts of acetate; sodium and potassium bicarbonates; lithium, sodium, potassium, and cesium carbonates; lithium, sodium, potassium, cesium, and magnesium chlorides; sodium and potassium fluorides; sodium, potassium, and calcium nitrates; sodium and potassium phosphates; and calcium and magnesium sulfates.

“Mobile phase modifiers” also include chaotropic agents, which weaken or otherwise interfere with non-covalent forces and increase entropy within biomolecular systems. Non-limiting examples of chaotropic agents include butanol, calcium chloride, ethanol, guanidinium chloride, lithium perchlorate, lithium acetate, magnesium chloride, phenol, propanol, sodium dodecyl sulfate, thiourea, and urea. Chaotropic agents include salts that affect the solubility of proteins. The more chaotropic anions include for example chloride, nitrate, bromide, chlorate, iodide, perchlorate, and thiocyanate. The more chaotropic cations include for example lithium, magnesium, calcium, and guanidinium.

“Mobile phase modifiers” include those moieties that affect ionic or other non-covalent interactions that, upon addition to a pH gradient or step, or upon equilibration of a Protein A support in a “mobile phase modifier” and application of a pH step or gradient, results in a broadening of pH unit distance between elution of a homodimeric IgG and a heterodimeric IgG (e.g., a wild-type human IgG and the same IgG but bearing one or more modifications of its CH3 domain as described herein). A suitable concentration of a “mobile phase modifier” can be determined by its concentration employing the same column, pH step or gradient, with increasing concentration of “mobile phase modifier” until a maximal pH distance is reached at a given pH step or pH gradient. “Mobile phase modifiers” may also include non-polar modifiers, including for example propylene glycol, ethylene glycol, and the like.

As used herein, “affinity chromatography” is a chromatographic method that makes use of the specific, reversible interactions between biomolecules rather than general properties of the biomolecule such as isoelectric point, hydrophobicity, or size, to effect chromatographic separation. “Protein A affinity chromatography” or “Protein A chromatography” refers to a specific affinity chromatographic method that makes use of the affinity of the IgG binding domains of Protein A for the Fc portion of an immunoglobulin molecule. This Fc portion comprises human or animal immunoglobulin constant domains CH2 and CH3 or immunoglobulin domains substantially similar to these. Protein A encompasses native protein from the cell wall of Staphylococcus aureus, Protein A produced by recombinant or synthetic methods, and variants that retain the ability to bind to an Fc region. In practice, Protein A chromatography involves using Protein A immobilized to a solid support. See Gagnon, Protein A Affinity Chromotography, Purification Tools for Monoclonal Antibodies, pp. 155-198, Validated Biosystems, 1996. Protein G and Protein L may also be used for affinity chromotography. The solid support is a non-aqueous matrix onto which Protein A adheres. Such supports include agarose, sepharose, glass, silica, polystyrene, nitrocellulose, charcoal, sand, cellulose and any other suitable material. Such materials are well known in the art. Any suitable method can be used to affix the second protein to the solid support. Methods for affixing proteins to suitable solid supports are well known in the art. See e.g. Ostrove, in Guide to Protein Purification, Methods in Enzymology, 182: 357-371, 1990. Such solid supports, with and without immobilized Protein A, are readily available from many commercial sources including such as Vector Laboratory (Burlingame, Calif.), Santa Cruz Biotechnology (Santa Cruz, Calif.), BioRad (Hercules, Calif.), Cytiva (Marlborough, Mass.), Pall (Port Washington, N.Y.) and EMD-Millipore (Billerica, Mass.). Protein A immobilized to a pore glass matrix is commercially available as PROSEP®-A (Millipore). The solid phase may also be an agarose-based matrix. Protein A immobilized on an agarose matrix is commercially available as MABSELECT™ (Cytiva.

Affinity chromatography also includes media that can be used to selectively bind and thus purify antibodies, fragments of antibodies, or chimeric fusion proteins that contain immunoglobulin domains and/or sequences. Antibodies include IgG, IgA, IgM, IgY, IgD and IgE types. Antibodies also include single chain antibodies such as camelid antibodies, engineered camelid antibodies, single chain antibodies, single-domain antibodies, nanobodies, and the like. Antibody fragments include VH, VL, CL, CH sequences. Antibody fragments and fusion proteins containing antibody sequences include for example F(ab′)₃, F(ab′)₂, Fab, Fc, Fv, dsFv, (scFv)₂, scFv, scAb, minibody, diabody, triabody, tetrabody, Fc-fusion proteins, trap molecules, and the like (see Ayyar et al., Methods 56 (2012): 116-129). Such affinity chromatography media may contain ligands that selectively bind antibodies, their fragments, and fusion proteins contains those fragments. Such ligands include antibody binding proteins, bacterially derived receptors, antigens, lectins or anti-antibodies directed to the target molecule (i.e., the molecule requiring purification). For example, camelid-derived affinity ligands directed against any one or more of IgG-CH1, IgG-Fc, IgG-CH3, IgG1, LC-kappa, LC-lambda, IgG3/4, IgA, IgM, and the like may be used as affinity ligands (commercially available as CAPTURESELECT chromatography resins, Life Technologies, Inc., Carlsbad, Calif.)

### Methods of Purifying Heterodimeric Proteins

Embodiments of methods of purifying heterodimeric proteins (via prolonging affinity column resolution and maintaining heterodimeric protein recovery rates) in accordance with the present disclosure include: (i) performing a preliminary series of cycles at a preliminary elution pH and a subsequent series of cycles at a subsequent (and higher) pH; (ii) performing a series of cycles in which the impurity level in the eluate is measured after each cycle, or periodically, and raising the elution pH in a subsequent cycle or cycles to maintain a minimal impurity level throughout the series of cycles; and (iii) performing a series of cycles in which the elution pH is raised in a step-wise manner over multiple sets of cycles (e.g., the elution pH is raised 0.5, 0.75 or 1 point following every 10, 15, 20, or 25 cycles).

Each of the methods of purifying a heterodimeric protein comprises: (a) performing a series of chromatographic cycles, wherein each cycle comprises: (i) introducing a mixture of a heterodimeric protein and impurities to an affinity matrix containing a protein-binding ligand, wherein the heterodimeric protein comprises first and second polypeptides with differing affinity for the protein-binding ligand, and wherein at least one impurity binds the protein-binding ligand and at least one impurity does not bind the protein-binding ligand; (ii) washing the affinity matrix with a first wash buffer at a first pH of from 5 to 9 to remove non-binding impurities; (iii) eluting the heterodimeric protein from the affinity matrix in a first elution buffer at a second pH; and (iv) washing the affinity matrix with a second wash buffer at a third pH of less than 4 to remove binding impurities, wherein the second pH is from 4.0 to 5.2.

In various embodiments, loading the mixture of heterodimeric protein and impurities onto the affinity matrix includes loading clarified cell culture from one or more bioreactors containing the cells expressing the nucleotide sequences encoding the heterdimeric protein. For example, the cells may express the nucleotides encoding each of the heavy and light chains forming a bispecific antibody. In some cases, each of the antigen-binding arms of the bispecific antibody comprises a common light chain. The clarified cell culture will include the heterodimeric protein (e.g., bispecific antibody), along with impurities such as homodimeric species, host cell proteins, and DNA. In some cases, the heterodimeric protein may be produced in eukaryotic cells, such as for example Chinese hamster ovary (CHO) cells.

In some embodiments, the mixture loaded onto the affinity matrix includes a mixture of proteins containing (i) a first homodimer comprising two copies of a first polypeptide, (ii) a heterodimer comprising the first polypeptide and a second polypeptide, and (iii) a second homodimer comprising two copies of the second polypeptide. The first and second polypeptides have different affinities for the affinity matrix, such that the first homodimer, the heterodimer and the second homodimer can be separated on the basis of differential binding to the affinity matrix. Differential binding to an affinity matrix can be manipulated by changing, inter alia, the pH and/or ionic strength of a solution passed over the affinity matrix.

Following loading of the clarified cell culture, the affinity matrix is washed with a wash buffer (first wash buffer) having a pH of from 5 to 9. In some cases, the pH of the wash buffer is from 6 to 8. In some cases, the pH of the wash buffer is from about 7 to about 7.5. In various embodiments, the pH of the wash buffer is or is about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 or 9.0. In some embodiments, the pH of the wash buffer is or is about 7.2. In various embodiments, the buffer can be any buffer capable of maintaining the pH at the desired point or within the desired range. In various embodiments, the buffer concentration may be from about 5 mM to about 100 mM. In some cases, the buffer concentration is from about 5 mM to about 15 mM. In some cases, the buffer concentration is from about 5 mM to about 50 mM. In some cases, the buffer concentration is from about 10 mM to about 25 mM. In some cases, the buffer concentration is from about 20 mM to about 40 mM. In some cases, the buffer concentration is from about 30 mM to about 50 mM. In various embodiments, the buffer concentration is or is about 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, or 50 mM. In some embodiments, the wash buffer concentration is or is about 10 mM. In some embodiments, the wash buffer concentration is or is about 40 mM. In some embodiments, the wash buffer is sodium phosphate. The wash buffer (first wash buffer) can also contain a salt as discussed below.

In some cases, the wash buffer comprises salt at a concentration of from about 200 mM to about 800 mM. In some cases, the wash buffer comprises salt at a concentration of from about 250 mM to about 750 mM. In some cases, the wash buffer comprises salt at a concentration of from about 300 mM to about 700 mM. In some cases, the wash buffer comprises salt at a concentration of from about 350 mM to about 650 mM. In some cases, the wash buffer comprises salt at a concentration of from about 400 mM to about 600 mM. In some cases, the wash buffer comprises salt at a concentration of from about 450 mM to about 550 mM. In some cases, the wash buffer comprises salt at a concentration of or of about 200 mM, 210, mM, 220 mM, 225 mM, 230 mM, 240 mM, 250 mM, 260 mM, 270 mM, 275 mM, 280 mM, 290 mM, 300 mM, 310 mM, 320 mM, 325 mM, 330 mM, 340 mM, 350 mM, 360 mM, 370 mM, 375 mM, 380 mM, 390 mM, 400 mM, 410 mM, 420 mM, 425 mM, 430 mM, 440 mM, 450 mM, 460 mM, 470 mM, 475 mM, 480 mM, 490 mM, 500 mM, 510 mM, 520 mM, 525 mM, 530 mM, 540 mM, 550 mM, 560 mM, 570 mM, 575 mM, 580 mM, 590 mM, 600 mM, 610 mM, 620 mM, 625 mM, 630 mM, 640 mM, 650 mM, 660 mM, 670 mM, 675 mM, 680 mM, 690 mM, 700 mM, 710 mM, 720 mM, 725 mM, 730 mM, 740 mM, 750 mM, 760 mM, 770 mM, 780 mM, 790 mM or 800 mM. In some embodiment, the salt concentration of the wash buffer is or is about 500 mM. In some embodiments, the wash buffer comprises about 500 mM NaCl. In some cases, this wash of the affinity matrix removes unbound impurities such as host cell protein, DNA and homodimeric species with little or no affinity for the affinity matrix material (e.g., Protein A).

In some embodiments, the methods include an optional second wash, prior to elution of the heterodimeric protein, with a wash buffer comprising little (<25 mM) or no salt at a pH of from 5 to 9. In some embodiments, this wash buffer comprises from about 10 mM to about 50 mM Tris [tris(hydroxymethyl)aminomethane]], sodium phosphate, or acetate, or combinations thereof. In various embodiments, this wash buffer has a pH that is equal to the pH of the first wash buffer discussed above.

Following the wash or washes discussed above, the heterodimeric protein is eluted from the affinity matrix in an elution buffer and collected in an eluate. The elution buffer has a pH of from about 4 to about 5.2 (or 4.0 to 4.9), and includes salt at a concentration of greater than 200 mM. As discussed in greater detail below in connection with the various methods, in some embodiments, the pH of the elution buffer is from about 4.0 to about 4.2. In some embodiments, the pH of the elution buffer is from about 4.4 to about 4.6. In various embodiments, the pH of the elution buffer is or is about 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95 or 5.0. In some embodiments, the pH of the elution buffer is 4.1±0.05. In some embodiments, the pH of the elution buffer is 4.5±0.05. In various embodiments, the buffer can be any buffer capable of maintaining the pH at the desired point or within the desired range. In various embodiments, the buffer concentration may be from about 5 mM to about 100 mM. In some cases, the buffer concentration is from about 25 mM to about 55 mM. In some cases, the buffer concentration is from about 30 mM to about 50 mM. In various embodiments, the buffer concentration is or is about 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, or 50 mM. In some embodiments, the elution buffer concentration is or is about 40 mM. In some embodiments, the elution buffer is acetic acid. In some embodiments, the elution buffer is acetate.

In some cases, the elution buffer comprises salt at a concentration of from about 200 mM to about 800 mM. In some cases, the elution buffer comprises salt at a concentration of from about 250 mM to about 750 mM. In some cases, the elution buffer comprises salt at a concentration of from about 300 mM to about 700 mM. In some cases, the elution buffer comprises salt at a concentration of from about 350 mM to about 650 mM. In some cases, the elution buffer comprises salt at a concentration of from about 400 mM to about 600 mM. In some cases, the elution buffer comprises salt at a concentration of from about 450 mM to about 550 mM. In some cases, the elution buffer comprises salt at a concentration of or of about 200 mM, 210, mM, 220 mM, 225 mM, 230 mM, 240 mM, 250 mM, 260 mM, 270 mM, 275 mM, 280 mM, 290 mM, 300 mM, 310 mM, 320 mM, 325 mM, 330 mM, 340 mM, 350 mM, 360 mM, 370 mM, 375 mM, 380 mM, 390 mM, 400 mM, 410 mM, 420 mM, 425 mM, 430 mM, 440 mM, 450 mM, 460 mM, 470 mM, 475 mM, 480 mM, 490 mM, 500 mM, 510 mM, 520 mM, 525 mM, 530 mM, 540 mM, 550 mM, 560 mM, 570 mM, 575 mM, 580 mM, 590 mM, 600 mM, 610 mM, 620 mM, 625 mM, 630 mM, 640 mM, 650 mM, 660 mM, 670 mM, 675 mM, 680 mM, 690 mM, 700 mM, 710 mM, 720 mM, 725 mM, 730 mM, 740 mM, 750 mM, 760 mM, 770 mM, 780 mM, 790 mM or 800 mM. In some embodiment, the salt concentration of the elution buffer is or is about 500 mM. In some embodiments, the elution buffer comprises about 500 mM NaCl. In some embodiments, the elution buffer comprises about 500 mM CaCl₂. In some embodiments, the elution buffer comprises about 500 mM MgCl₂.

Following elution and collection of the heterodimeric protein from the affinity matrix, the affinity matrix is washed with a wash buffer (second wash buffer) at a pH of less than about 4. In some embodiments, the pH of the wash buffer is from about 2.5 to about 3.5. In some embodiments, the pH of the wash buffer is 3.0±0.2. In various embodiments, the pH of the wash buffer is or is about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8 or 3.9. The wash buffer can comprise any suitable material to provide the pH or range of pH noted above. In some embodiments, the wash buffer comprises acetic acid at a concentration of from about 20 mM to about 60 mM. In some embodiments, the wash buffer comprises acetic acid at a concentration of from about 30 mM to about 50 mM. In some cases, the wash buffer comprises about 40 mM acetic acid. In some cases, this wash of the affinity matrix removes formerly bound impurities such as homodimeric species with greater affinity for the affinity matrix material (e.g., Protein A) than the heterodimeric protein. In some cases, the methods of the present invention may also include a further wash of the affinity matrix with a buffer comprising a lower pH (e.g. 2.45±0.2) and a higher concentration of the buffer material (e.g. 500 mM acetic acid) than the wash buffer discussed immediately above.

Following removal of additional impurities with the wash (or washes) discussed above, the affinity matrix may be re-equilibrated to a pH of from 5 to 9 before beginning the next cycle. In various embodiments, the affinity matrix is equilibrated to a pH of or of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 or 9.0. In some embodiments, the affinity matrix is equilibrated to a pH of about 7.2. Equilibration can be performed with an equilibration buffer having the desired pH. In various embodiments, the buffer can be any buffer capable of maintaining the pH at the desired point or within the desired range. In various embodiments, the buffer concentration may be from about 5 mM to about 100 mM. In some cases, the buffer concentration is from about 10 mM to about 30 mM. In some cases, the buffer concentration is from about 30 mM to about 50 mM. In some cases, the buffer concentration is from about 40 mM to about 60 mM. In various embodiments, the buffer concentration is or is about 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM or 60 mM. In some embodiments, the buffer concentration is or is about 20 mM. In some embodiments, the buffer concentration is or is about 40 mM. In some embodiments, the buffer concentration is or is about 50 mM. In some embodiments, the buffer is sodium phosphate. In some embodiments, this buffer comprises from about 10 mM to about 50 mM Tris, sodium phosphate, or acetate, or combinations thereof.

Following equilibration of the affinity matrix, the neutralized eluate containing the heterodimeric protein (now purified from the homodimeric contaminants and other impurities) is reapplied to the same affinity matrix used in the purification process steps discussed above at a pH of from 5 to 9. In various embodiments, the neutralized eluate is reapplied to the affinity matrix at a pH of or of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 or 9.0. In some embodiments, the pH is or is about 7.2.

In some embodiments, a method of purifying a heterodimeric protein comprises: (a) performing a series of chromatographic cycles, wherein each cycle comprises: (i) introducing a mixture of a heterodimeric protein and impurities to an affinity matrix containing a protein-binding ligand, wherein the heterodimeric protein comprises first and second polypeptides with differing affinity for the protein-binding ligand, and wherein at least one impurity binds the protein-binding ligand and at least one impurity does not bind the protein-binding ligand; (ii) washing the affinity matrix with a first wash buffer at a first pH of from 5 to 9 to remove non-binding impurities; (iii) eluting the heterodimeric protein from the affinity matrix in a first elution buffer at a second pH; and (iv) washing the affinity matrix with a second wash buffer at a third pH of less than 4 to remove binding impurities, wherein the second pH is at a preliminary pH during a preliminary series of cycles within the series of chromatographic cycles, and the second pH is raised to a subsequent pH higher than the preliminary pH during a subsequent series of cycles within the series of chromatographic cycles, wherein the preliminary pH and the subsequent pH are within a range of from 4.0 to 5.2; and (b) collecting the heterodimeric protein from the affinity matrix in an eluate.

In various embodiments, the preliminary series of cycles consists of 20 cycles. In some embodiments, the preliminary series of cycles consists of 30 cycles. In some embodiments, the preliminary series of cycles consists of 40 cycles. In some embodiments, the preliminary series of cycles consists of 50 cycles. In some embodiments, the preliminary series of cycles consists of 60 cycles. In some embodiments, the preliminary series of cycles consists of 70 cycles. In some embodiments, the preliminary series of cycles consists of 80 cycles. In some cases, the preliminary series of cycles comprises, or consists of, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 cycles, or more.

In some embodiments, the subsequent series of cycles consists of at least 20 cycles. In some embodiments, the subsequent series of cycles consists of at least 50, at least 60, at least 70, or at least 80 cycles. In some cases, the subsequent series of cycles comprises, or consists of, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 85, 90, 95 or 100 cycles, or more.

In some embodiments, the preliminary pH is from 4.0 to 4.2. In some cases, the preliminary pH is 4.1±0.05. In some cases, the preliminary pH is 4.0, 4.025, 4.05, 4.075, 4.1, 4.125, 4.15, 4.175, or 4.2. In some embodiments, the subsequent pH is from 4.3 to 4.7. In some cases, the subsequent pin some cases, the subsequent pH is 4.5±0.05. In some cases, the subsequent pH is 4.4, 4.425, 4.45, 4.475, 4.5, 4.525, 4.55, 4.575, or 4.6.

In some embodiments, a method of purifying a heterodimeric protein comprises: (a) performing a series of chromatographic cycles, wherein each cycle comprises: (i) introducing a mixture of a heterodimeric protein and impurities to an affinity matrix containing a protein-binding ligand, wherein the heterodimeric protein comprises first and second polypeptides with differing affinity for the protein-binding ligand, and wherein at least one impurity binds the protein-binding ligand and at least one impurity does not bind the protein-binding ligand; (ii) washing the affinity matrix with a first wash buffer at a first pH of from 5 to 9 to remove non-binding impurities; (iii) eluting the heterodimeric protein from the affinity matrix in a first elution buffer at a second pH; and (iv) washing the affinity matrix with a second wash buffer at a third pH of less than 4 to remove binding impurities; (b) measuring a level of binding impurity in an eluate containing the heterodimeric protein following any one or more of the cycles within the series of chromatographic cycles, and comparing the measured level of binding impurity to a reference level of binding impurity, wherein if the measured level of binding impurity exceeds the reference level of binding impurity, then increasing the second pH in a subsequent cycle within the series of chromatographic cycles, wherein the second pH is within a range of from 4.0 to 5.2 during each cycle or subsequent cycle within the series of chromatographic cycles; and (c) collecting the heterodimeric protein from the affinity matrix in the eluate.

In some embodiments, the reference level of binding impurity is from 2% to 10%. In some cases, the reference level of binding impurity is from 3% to 7%. In some cases, the reference level of binding impurity is 5%±0.5%. In various embodiments, the reference level of binding impurity is, or is about, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%.

In some embodiments, the level of binding impurity in the eluate is measured following each cycle within the series of chromatographic cycles. In some embodiments, the level of binding impurity in the eluate is measured following every fifth cycle in the series of chromatographic cycles. In some embodiments, the level of binding impurity in the eluate is measured following every tenth cycle in the series of chromatographic cycles. In some embodiments, the level of binding impurity in the eluate is measured following a twentieth cycle in the series of chromatographic cycles. In some embodiments, the level of binding impurity in the eluate is measured following a fortieth cycle or a fiftieth cycle in the series of chromatographic cycles. In various embodiments, the level of binding impurity in the eluate is measured after cycle 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or cycle 100. In various embodiments, the level of binding impurity in the eluate is measured after every 2 cycles, every 3 cycles, every 4 cycles, every 5 cycles, every 6 cycles, every 7 cycles, every 8 cycles, every 9 cycles, every 10 cycles, every 15 cycles, every 20 cycles, every 25 cycles, every 30 cycles, every 35 cycles, every 40 cycles, every 45 cycles, or every 50 cycles. In some cases, the eluate is collected over a series of cycles (e.g., five cycles, or ten cycles), and the level of binding impurity is measured in the combined eluate pool. In various embodiments, the combined eluate pool is collected over a series of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 cycles, or more.

In some embodiments, the second pH is increased to a range of from 4.2 to 5.2 (or from 4.3 to 4.7) from a range of from 4.0 to 4.2 if the measured level of binding impurity exceeds the reference level of binding impurity. In some cases, the second pH is increased to 4.5±0.05 from 4.1±0.05 if the measured level of binding impurity exceeds the reference level of binding impurity. In some cases, the second pH is 4.0, 4.025, 4.05, 4.075, 4.1, 4.125, 4.15, 4.175, or 4.2, and is increased to 4.4, 4.425, 4.45, 4.475, 4.5, 4.525, 4.55, 4.575, or 4.6 if the measured level of binding impurity exceeds the reference level of binding impurity.

In some embodiments, the second pH is 4.0 to 4.2, and is increased by 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75 or 5 points in the subsequent cycles if the measured level of binding impurity exceeds the reference level of binding impurity. Thus, in some cases, the second pH may be incrementally increased in a subsequent cycle if the measured level of binding impurity exceeds the reference level of binding impurity, and then incrementally increased again (and again, and again, etc., as necessary) if the measured level of binding impurity exceeds the reference level of binding impurity in the next cycle for which a measurement is made. In this manner, the elution pH can be maintained at a level that provides minimal binding impurity in the eluate while maintaining maximum recovery of the heterodimeric protein over the course of a series of chromatographic cycles.

In some embodiments, a method of purifying a heterodimeric protein comprises: (a) performing a series of chromatographic cycles, wherein each cycle comprises: (i) introducing a mixture of a heterodimeric protein and impurities to an affinity matrix containing a protein-binding ligand, wherein the heterodimeric protein comprises first and second polypeptides with differing affinity for the protein-binding ligand, and wherein at least one impurity binds the protein-binding ligand and at least one impurity does not bind the protein-binding ligand; (ii) washing the affinity matrix with a first wash buffer at a first pH of from 5 to 9 to remove non-binding impurities; (iii) eluting the heterodimeric protein from the affinity matrix in a first elution buffer at a second pH; and (iv) washing the affinity matrix with a second wash buffer at a third pH of less than 4 to remove binding impurities; wherein the second pH is at a primary pH during a primary series of cycles within the series of chromatographic cycles, the second pH is raised to a secondary pH higher than the primary pH during a secondary series of cycles that succeeds the primary series of cycles within the series of chromatographic cycles, and the second pH is raised to a tertiary pH higher than the secondary pH during a tertiary series of cycles that succeeds the secondary series of cycles within the series of chromatographic cycles, wherein the primary pH, the secondary pH, and the tertiary pH are within a range of from 4.0 to 5.2; and (b) collecting the heterodimeric protein from the affinity matrix in an eluate.

In some embodiments, the primary series of cycles comprises from 5 to 50 cycles. In some cases, the primary series of cycles comprises up to 20 cycles. In some cases, the primary series of cycles comprises up to 40 cycles. In some cases, the primary series of cycles includes, or includes up to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70 or 75 cycles, or more.

In some embodiments, the secondary series of cycles comprises from 5 to 50 cycles. In some cases, the secondary series of cycles comprises from 10 to 25 cycles. In some cases, the secondary series of cycles includes, or includes up to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70 or 75 cycles, or more.

In some embodiments, the tertiary series of cycles comprises from 5 to 50 cycles. In some cases, the tertiary series of cycles comprises from 10 to 25 cycles. In some cases, the tertiary series of cycles includes, or includes up to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70 or 75 cycles, or more.

In some embodiments, the primary pH is in a range of from 4.0 to 4.2. In some cases, the primary pH is 4.1±0.05. In some cases, the primary pH is 4.0, 4.025, 4.05, 4.075, 4.1, 4.125, 4.15, 4.175, or 4.2. In some embodiments, the secondary pH is in a range of from 4.2 to 4.4. In some cases, the secondary pH is 4.3±0.05. In some cases, the secondary pH is 4.2, 4.225, 4.25, 4.275, 4.3, 4.325, 4.35, 4.375, or 4.4. In some embodiments, the tertiary pH is in a range of from 4.4 to 4.6. In some cases, the tertiary pH is 4.5±0.05. In some cases, the tertiary pH is 4.4, 4.425, 4.45, 4.475, 4.5, 4.525, 4.55, 4.575, or 4.6.

In some embodiments, the second pH is raised to a 4th pH higher than the tertiary pH during a 4th series of cycles that succeeds the tertiary series of cycles within the series of chromatographic cycles, wherein the 4th pH is within a range of from 4.0 to 5.2.

In some embodiments, the second pH is raised to a 5th pH higher than the 4th pH during a 5th series of cycles that succeeds the 4th series of cycles within the series of chromatographic cycles, wherein the 5th pH is within a range of from 4.0 to 5.2.

In some embodiments, the second pH is raised to a 6th pH higher than the 5th pH during a 6th series of cycles that succeeds the 5th series of cycles within the series of chromatographic cycles, wherein the 6th pH is within a range of from 4.0 to 5.2.

In some cases, the secondary pH is a pH from 0.1 to 0.9 higher than the primary pH, the tertiary pH is a pH from 0.1 to 0.9 higher than the secondary pH, the 4th pH is a pH from 0.1 to 0.9 higher than the tertiary pH, the 5th pH is a pH from 0.1 to 0.9 higher than the 4th pH, and/or the 6th pH is a pH from 0.1 to 0.9 higher than the 5th pH, wherein the primary pH is in a range of from 4.0 to 4.2. In some embodiments, the primary pH is 4.1±0.05.

In some embodiments, the secondary, tertiary, 4th, 5th or 6th pH (or 7th, 8th, 9th, etc. pH) is increased by 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1, 1.25, 1.5, 1.75, 2, 2.25, 2.5, 2.75, 3, 3.25, 3.5, 3.75, 4, 4.25, 4.5, 4.75 or 5 points from the immediately preceding pH (e.g., the secondary pH is increased relative to the primary pH, and the tertiary pH is increased relative to the secondary pH, etc.) in the next series of cycles. Thus, in some cases, the elution pH may be incrementally increased in each succeeding series of cycles. In this manner, the elution pH can be maintained at a level that provides minimal binding impurity in the eluate while maintaining maximum recovery of the heterodimeric protein over the course of a series of chromatographic cycles.

In some embodiments, each of the primary series of cycles, the secondary series of cycles, the tertiary series of cycles, the 4th series of cycles, the 5th series of cycles, and/or the 6th series of cycles (or further series if desired) comprises from 5 to 50 cycles within the series of chromatographic cycles. In various embodiments, each series of cycles includes, or includes at least, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70 or 75 cycles, or more.

In various embodiments, loading of the affinity matrix from clarified cell culture or from the neutralized eluate containing the heterodimeric protein can include addition of material of up to about 75 g/L of affinity matrix resin. In various embodiments, the affinity matrix is loaded with less than or equal to 65 g/L, 60 g/L, 55 g/L or 50 g/L of material.

In some embodiments, the affinity matrix comprises a ligand (e.g., Protein A) affixed to a substrate. In some cases, the substrate is a bead or particle, such that the affinity matrix is a plurality of particles affixed with the ligand. In various embodiments, the ligand is Protein A or Protein G. When the ligand is Protein A, the Protein A may be a naturally occurring or modified Staphylococcal Protein A, or it may be an engineered Protein A. Engineered Protein A may be for example a Z-domain tetramer, a Y-domain tetramer, or an engineered Protein A that lacks D and E domains. These engineered Protein A exemplars are unable to bind (or bind with very low affinity if at all) to the VH3 domain of an immunoglobulin, but can still bind to the CH3 domains of IgG1, IgG2 and IgG4.

In some cases, the affinity matrix substrate contains or is made of agarose, poly(styrene divinylbenzene), polymethacrylate, controlled pore glass, spherical silica, cellulose and the like. In the embodiments in which the substrate is shaped as a bead or particle, the mean diameter of the particles is from 25 μm to 100 μm. In some embodiments, the mean diameter of the particles is from about 40 μm to about 60 μm. In some embodiments, the mean diameter of the particles is from about 45 μm to about 55 μm. In some embodiments, the mean diameter of the particles is from about 40, 41, 42, 43, 44, 45, 46, 47, 48, 49, 50, 51, 52, 53, 54 or 55 μm. In some cases, the mean diameter of the particles is about 45 μm. In some cases, the mean diameter of the particles is about 50 μm. In some embodiments, the particles have a mean diameter of 35 μm, 45 μm, 60 μm, 75 μm, or 85 μm. In some embodiments, the particles contain pores having a mean diameter of about 1000 Å, 1050 Å, 1100 Å, 1150 Å or 1200 Å. In some embodiments, the particles contain pores having a mean diameter of about 1100 Å.

In various embodiments, the elution buffer or wash buffers may comprise a salt. In some cases, the salt comprises Cl⁻, Br⁻, I⁻, NO₃⁻, N(CH₃)₄⁺, NH₄⁺, Cs⁺, Rb⁺, K⁺, Na⁺, H⁺, Ca²⁺, Mg²⁺, Al³⁺. In some embodiments, the salt comprises Na⁺, H⁺, Ca²⁺, Mg²⁺ or Al³⁺. In some embodiments, the salt comprises Cl⁻, Br⁻, I⁻, NO₃⁻, or ClO₄⁻. In some embodiments, the salt comprises combinations of Na⁺, H⁺, Ca²⁺, Mg²⁺ or Al³⁺ with Cl⁻, Br⁻, I⁻, NO₃⁻, or ClO₄⁻. In some embodiments, the salt is selected from CaCl₂, MgCl₂ or NaCl. In some embodiments, the salt is NaCl. In some embodiments, the salt is CaCl₂. In some embodiments, the salt is MgCl₂.

In some embodiments of the methods, the heterodimeric protein is a bispecific antibody comprising a first polypeptide comprising a CH3 domain that is capable of binding to Protein A (“Fc”) and a second polypeptide comprising a CH3 domain that is not capable of binding to Protein A (“Fc*”). In some cases, the second polypeptide comprises a H435R/Y436F (by EU numbering system; H95R/Y96F by IMGT exon numbering system) substitution in its CH3 domain (a.k.a “Fc*” or “star substitution”). Thus, in some embodiments, the first homodimer is a monospecific antibody having two unsubstituted CH3 domains (i.e., FcFc); the second homodimer is a monospecific antibody having two H435R/Y436F substituted CH3 domains (i.e., Fc*Fc*); and the heterodimeric protein is a bispecific antibody having one unsubstituted CH3 domain and one H435R/Y436F substituted CH3 domain (i.e., Fc*Fc).

In some embodiments of the methods, the frequency with which the chromatography column is subjected to cleaning (e.g., by contacting the column with a basic solution having a pH of at least 11) can be reduced in order to minimize impacts on protein-ligand function. Similarly, in some embodiments of the methods, the concentration of the base in the solution used for cleaning the chromatography column can be reduced to a range of from 0.1 N to 0.5 N in order to maximize column resolution over a larger number of cycles.

In various embodiments, the affinity matrix may be contacted with a basic solution having a pH of at least 11 following every cycle. In some cases, the affinity matrix is contacted with a basic solution having a pH of at least 11 following every three cycles. In some cases, the affinity matrix is contacted with a basic solution having a pH of at least 11 following every five cycles. In some cases, the affinity matrix is contacted with a basic solution having a pH of at least 11 following every seven cycles. In various embodiments, the affinity matrix is contacted with a basic solution having a pH of at least 11 following only every 2 cycles, every 3 cycles, every 4 cycles, every 5 cycles, every 6 cycles, every 7 cycles, every 8 cycles, every 9 cycles, or every 10 cycles.

In some embodiments, the pH of the basic solution is at least 12. In some embodiments, the pH of the basic solution is at least 11, at least 11.1, at least 11.2, at least 11.3, at least 11.4, at least 11.5, at least 11.6, at least 11.7, at least 11.8, at least 11.9, at least 12, at least 12.1, at least 12.2, at least 12.3, at least 12.4, at least 12.5, at least 12.6, at least 12.7, at least 12.8, at least 12.9, or at least 13.

In some embodiments, the basic solution comprises a base at a concentration of from 0.1 N to 0.5 N. In some cases, the base concentration is from 0.1 N to 0.3 N. In some case, the base concentration is 0.1 N, 0.15 N, 0.2 N, 0.25 N, 0.3 N, 0.35 N, 0.4 N, 0.45 N, or 0.5 N. In some embodiments, the basic solution comprises an alkali metal hydroxide. In some cases, the base is NaOH. In some cases, the base is KOH.

### EXAMPLES

### Example 1: Evaluation of Elution pH on the Presence of Binding Impurity and the Recovery Rate of the Heterodimeric Protein in Affinity Chromatography

A 16.2 mL MabSelect SuRe™ pcc column (1.0 cm i.d., 20.6 cm bed height) was packed with naïve resin and integrated onto an AKTA Avant 25 bench top liquid chromatography controller for this experiment. The affinity resolving process was conducted as outlined in Table 1, below, but with varying elution pH of from 3.90 to 4.30.

TABLE 1
Process for Elution Buffer Determination for bsAb1 Affinity Resolving
Chromatography with MabSelect SuRe ™ pcc

| | | | | Residence |
|---|---|---|---|---|
| | | | | Time |
| Step | Description | Solution | Volume | (min) |
| 1 | Remove Storage | RODI | 2 CV | 10 |
| | Ethanol | | | |
| 2 | Pre-Strip | 500 mM Acetic Acid, pH 2.45 ± 0.20 | 2 CV | 6 |
| 3 | Equilibration | 40 mM Sodium Phosphate, 500 mM NaCl, | 2 CV | 6 |
| | | pH 7.20 ± 0.10 | | |
| 4 | Load | Clarified Cell Culture | 55.0 g | 6 |
| | | | binding | |
| | | | species/L | |
| | | | resin^{a} | |
| 5 | Wash 1 | 40 mM Sodium Phosphate, 500 mM NaCl, | 3 CV | 6 |
| | | pH 7.20 ± 0.10 | | |
| 6 | Wash 2 | 40 mM Tris, 10 mM Acetate, | 2 CV | 6 |
| | | pH 7.20 ± 0.10 | | |
| 7 | Elution | 40 mM Acetate, 500 mM NaCl | 6 CV^{b} | 6 |
| | | pH 4.10 ± 0.05 | | |
| 8 | Strip 1 | 40 mM Acetic Acid, | 2 CV | 6 |
| | | pH 3.00 ± 0.10 | | |
| 9 | Strip 2 | 500 mM Acetic Acid, | 2 CV | 6 |
| | | pH 2.45 ± 0.20 | | |
| 10 | Re-Equilibration | 20 mM Sodium Phosphate, | 2 CV | 6 |
| | | pH 7.20 ± 0.10 | | |
| 11 | Storage | 20% (v/v) Ethanol | 2 CV | 10 |

^{a}Binding species refers to the bispecific and binding impurity species. Binding titer was used to determine column loading.
^{b}Eluate collection began 0.5 CV into elution block.
CV, column volume;
RODI, reverse osmosis deionized water

Affinity resolving eluates were fractionated to enable preparation of mock pools representing eluate composition at elution lengths of 5, 6, and 7 CVs. Eluate collection began 0.5 CVs into the elution block. CVs 0.5-5 were collected in bulk, followed by individual collections of CV 5-6 and CV 6-7. Following fractionation, appropriate volumes were combined from each fraction to generate 6 CV and 7 CV mock pools; 5, 6, and 7 CV pools were then statistically evaluated as discrete runs.

Concentration of each mock pool was determined by UV (280 nm) with a Solo VPE instrument. Each mock pool was analyzed for bispecific purity measured using a mixed-mode chromatography assay. Eluate volume, eluate protein concentration, binding impurity, and non-binding impurity data of each mock pool were used to calculate affinity resolving bispecific yield for each run. Models were generated using factors selected from a backwards stepwise regression tool with a 0.25 probability to enter, 0.05 probability to leave, and a p-value threshold stopping rule set to 95%, and used to calculate binding impurity levels and heterodimeric protein recovery rates.

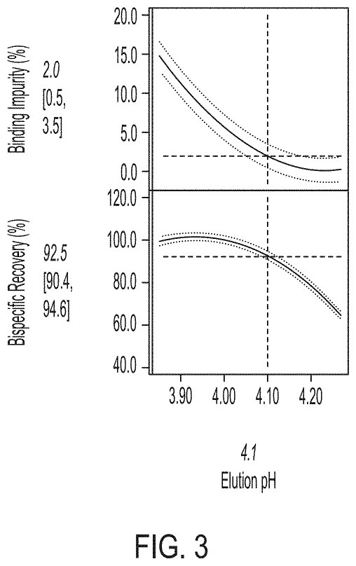

As shown in FIG. 3, both the percentage of binding impurity in the eluate and the percentage of heterodimeric protein recovery decreased with increasing pH in the naïve column (0 prior cycles). As shown, a pH of 4.1 provides a minimal level of binding impurity in the eluate (e.g., 2.0%), while maintaining a significant level of heterodimeric protein recovery (e.g., 92.5%). Notably, raising the pH of the elution buffer to even 4.2 dramatically reduces the recovery rate of the heterodimeric protein (e.g., to about 80%).

### Example 2: Evaluation of Elution pH on the Presence of Binding Impurity and the Recovery Rate of the Heterodimeric Protein in Naive and Cycled Affinity Chromatography Columns

A MabSelect SuRe™ pcc column (1.0 cm inner diameter, 20 cm bed height) integrated into an Akta Avant 25 (Cytiva) liquid chromatography system was used to perform this experiment. The affinity resolving process was conducted as outlined in Table 2, below, but with varying elution pH and cycles numbers, as shown in Table 3, below.

TABLE 2
Process for Elution Buffer Determination for bsAb1 Affinity Resolving
Chromatography with MabSelect SuRe ™ pcc in Naïve and Cycled Columns

| | | | | Residence |
|---|---|---|---|---|
| | | | | Time |
| Step | Description | Solution | Volume | (min) |
| 1 | Remove Storage | Reverse Osmosis Deionized Water | 2 CV | 10 |
| | Ethanol | | | |
| 2 | Pre-Strip | 500 mM Acetic Acid, pH 2.45 ± 0.20 | 2 CV | 6 |
| 3 | Equilibration | 20 mM Sodium Phosphate, | 2 CV | 6 |
| | | pH 7.20 ± 0.10 | | |
| 4 | Load | Clarified Cell Culture | 52-63 g | 6 |
| | | | binding | |
| | | | species | |
| | | | (binding | |
| | | | impurity + | |
| | | | bispecific) | |
| | | | per L resin | |
| 5 | Wash 1 | 10 mM Sodium Phosphate, 525 mM | 3 CV | 6 |
| | | NaCl, | | |
| | | pH 7.10 ± 0.10 | | |
| 6 | Wash 2 | 20 mM Sodium Phosphate, | 2 CV | 6 |
| | | pH 7.20 ± 0.10 | | |
| 7 | Elution | 40 mM Acetate, | ≤7.25 | 8 |
| | | 500 mM NaCl, | CV | |
| | | pH 4.10 ± 0.05 | | |
| | | OR | | |
| | | 40 mM Acetate, | | |
| | | 500 mM NaCl, | | |
| | | pH 4.50 ± 0.05 | | |
| 8 | Strip 1 | 40 mM Acetic Acid, | 2 CV | 6 |
| | | 9 mM NaCl, | | |
| | | pH 3.10 ± 0.10 | | |
| 9 | Strip 2 | 500 mM Acetic Acid, | 2 CV | 6 |
| | | pH 2.45 ± 0.20 | | |
| 10 | Re-Equilibration | 20 mM Sodium Phosphate, | 2 CV | 6 |
| | | pH 7.20 ± 0.10 | | |
| 11 | Strip 3 | 0.5M NaOH | 2 CV | 7.5 |
| 12 | Re-Equilibration | 20 mM Sodium Phosphate, | 2 CV | 6 |
| | | pH 7.20 ± 0.10 | | |
| 13 | Storage | 20% (v/v) Ethanol | 2 CV | 10 |

TABLE 3
pH and Cycle Number of Resin

| Run | Cycle number on resin |
|---|---|
| 4.1 naive | 1 |
| 4.1 cycled | 78 |
| 4.5 naïve | 6 |
| 4.5 cycled | 83 |

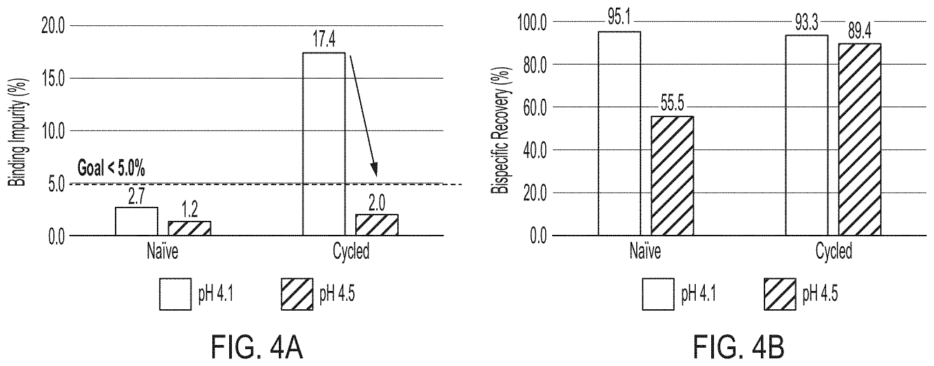

As shown in FIG. 4A, increasing the elution pH (from 4.1 to 4.5) in a naïve column (6 cycles) slightly reduces the percentage of binding impurity in the eluate (from 2.7% to 1.2%), whereas increasing the elution pH (from 4.1 to 4.5) in a cycled column (78-83 cycles) dramatically and unexpectedly reduces the percentage of binding impurity in the eluate (from 17.4% to 2.0%). FIG. 4B shows that the increase in the elution pH (from 4.1 to 4.5) also negatively impacts the recovery percentage of the heterodimeric protein (e.g., a bispecific antibody), but the reduction in recovery percentage in a cycled column is unexpectedly much less significant (˜10× less relative to a naïve column). As shown in FIG. 4B, heterodimeric protein recovery was reduced by ˜40% in a naïve column when the elution pH was raised from 4.1 to 4.5, whereas the reduction in a cycled column was only ˜4% for the same pH increase.

### Example 3: Evaluation of Input Parameters on Measured Outputs in a pH Elution Study

Three MabSelect SuRe™ pcc columns (1.0 cm inner diameter, 21 cm bed height; 16.5 mL column volume) individually integrated into an AKTA pure 150 (Cytiva) liquid chromatography system were used to perform this experiment. The affinity resolving process was conducted as outlined in Table 4, below, but with varying column loading (33-55 g of binding species (bispecific +binding impurity) per L of resin), elution pH (4.0-4.5), and hydroxide cycles or hydroxide exposure time (1-109 cycles or 0.28-30.56 hours), as shown in Table 5, below. Yield (% of bispecific +binding impurity), binding impurity (%), and aggregation (SE-UPLC high molecular weight %) were measured in connection with column loading, elution pH, and hydroxide cycles (or exposure time).

TABLE 4
Process for pH Elution Study of bsAb1

| | | | | Residence |
|---|---|---|---|---|
| Step | Description | Solution | Volume | Time (min) |
| 1 | Water Flush | Purified Water | 3 CV | 31.5 |
| 2 | Pre-Strip | 500 mM Acetic Acid, pH 2.45 ± 0.2 | 2 CV | 12.6 |
| 3 | Equilibration | 20 mM Sodium Phosphate, pH 7.20 ± 0.1 | 2 CV | 12.6 |
| 4 | Load | Clarified Cell Culture | See Table | 51.4-85.7 |
| | | | x | |
| 5 | Wash 1 | 10 mM Sodium Phosphate, 500 mM | 3 CV | 18.9 |
| | | Sodium Chloride, pH 7.20 ± 0.1 | | |
| 6 | Wash 2 | 20 mM Sodium Phosphate, pH 7.20 ± 0.1 | 2 CV | 12.6 |
| 7 | Elution | 40 mM Acetate, 500 mM Sodium Chloride | 6 CV | 37.8 |
| | | Solution pH: See Table 5 | | |
| 8 | Strip 1 | 40 mM Acetic Acid, pH 3.00 ± 0.2 | 2 CV | 12.6 |
| 9 | Strip 2 | 500 mM Acetic Acid, pH 2.45 ± 0.2 | 2 CV | 12.6 |
| 10 | Re-Equilibration | 20 mM Sodium Phosphate, pH 7.20 ± 0.1 | 2 CV | 12.6 |

TABLE 5
Variation of Parameters in Experimental Runs

| | | | | Hydroxide |
|---|---|---|---|---|
| | | Loading | Hydroxide | Contact Time |
| Run | Resolving pH | (g/L resin) | cycles | (hr) |
| 1 | 4.00 | 55 | 105 | 29.44 |
| 2 | 4.50 | 33 | 1 | 0.28 |
| 3 | 4.00 | 33 | 2 | 0.56 |
| 4 | 4.25 | 44 | 106 | 29.72 |
| 5 | 4.25 | 44 | 50 | 14.02 |
| 6 | 4.50 | 55 | 3 | 0.84 |
| 7 | 4.00 | 55 | 4 | 1.12 |
| 8 | 4.25 | 33 | 51 | 14.30 |
| 9 | 4.25 | 55 | 52 | 14.58 |
| 10 | 4.25 | 44 | 53 | 14.86 |
| 11 | 4.50 | 44 | 54 | 15.14 |
| 12 | 4.25 | 44 | 55 | 15.42 |
| 13 | 4.50 | 55 | 107 | 30.00 |
| 14 | 4.00 | 33 | 108 | 30.28 |
| 15 | 4.00 | 44 | 56 | 15.70 |
| 16 | 4.25 | 44 | 5 | 1.40 |
| 17 | 4.50 | 33 | 109 | 30.56 |

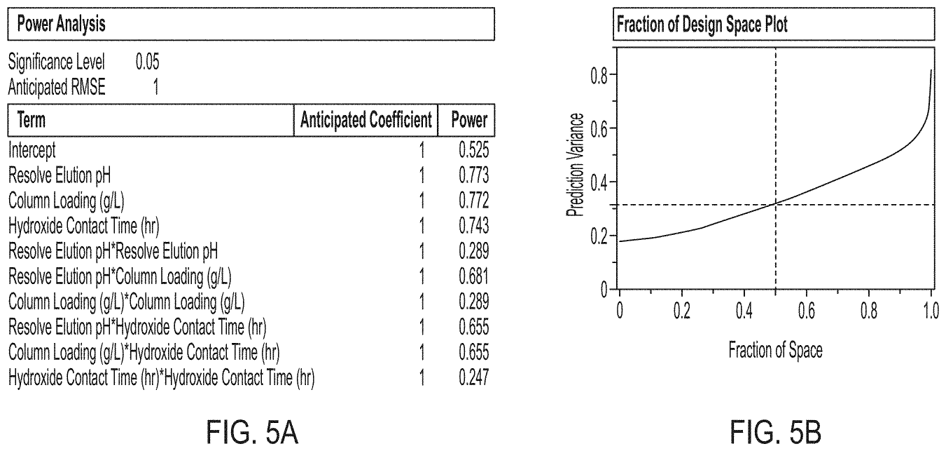

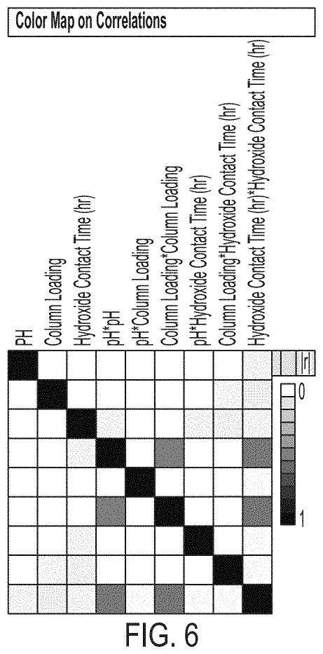

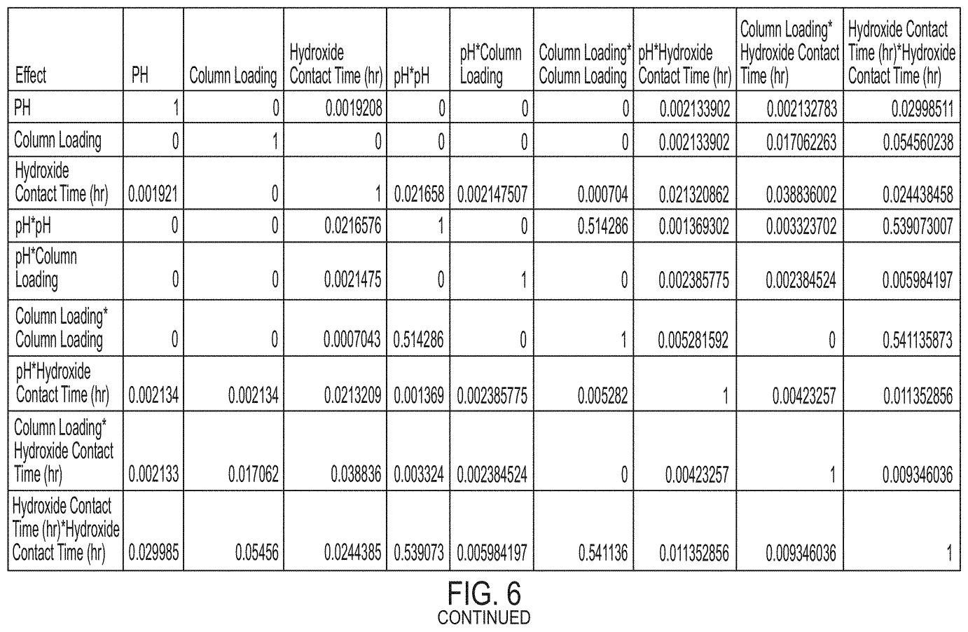

The experimental runs were executed in the order listed above in Table 5. The design diagnostics are presented in FIGS. 5A, 5B and 6. The experiments were conducted across three columns, as noted above, that had low (1-5), moderate (50-56) or high (105-109) numbers of hydroxide cycles. The hydroxide cycles were converted to hydroxide cycle time to facilitate analysis. The hydroxide contact time was 16.82 min (0.28 hr) per cycle. The resolving elution buffers were prepared within a pH tolerance of ±0.05. The eluate collection began 0.5 column volumes (CV) into elution block.

Concentration of each pool was determined by UV (280 nm) with a Solo VPE instrument. Eluate volume and eluate protein concentration were used to calculate affinity resolving bispecific yield for each run assuming the pool only contained bispecific protein (i.e., due to impurities (binding impurity), the resulting yield could be measured at >100%). Bispecific purity was measured using a hydrophobic interaction chromatography (HIC) assay. Aggregation was measured using a size exclusion ultra-high performance liquid chromatography assay (SE-UPLC).

Models were generated using factors selected from a backwards stepwise regression tool starting with the full model, combine rule and a p-value threshold of 0.05 to leave. Process knowledge and/or further statistical analysis was also used to add or remove model terms when appropriate. Regression analysis was performed for bispecific step yield (%), binding impurity (%) and aggregation (% HMVV).

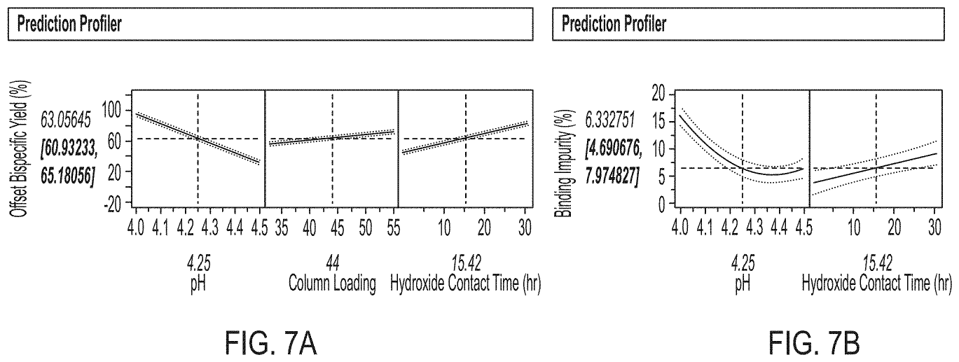

Model prediction profilers are shown in FIGS. 7A and 7B. Models with significant terms were created for bispecific yield and binding impurity. No significant terms were found for aggregation. Lower pH and higher column loading, and hydroxide contact time produced higher bispecific yield. A main component of this higher yield was due to the presence of increased binding impurity, as shown in FIG. 7B, which followed the same trends for pH and hydroxide contact time.

Three confirmation runs were performed to assess the capability of these models to predict new data. Clarified cell culture was purified on each of the three columns using an average column loading of 44 g/L of resin. The models were used to predict what pH should be used to target a fixed binding impurity level (˜6%) on columns at different stages of their resin lifetime. The results are presented below in Table 6.

TABLE 6
Model Confirmation Runs

| | | | | | Resolve | Resolve | Binding | Binding |
|---|---|---|---|---|---|---|---|---|
| | Resolving | Loading | | Hydroxide | Bispecific Step | Bispecific | Impurity | Impurity |
| | Elution | (g/L of | Hydroxide | Contact | Yield | Step Yield | (%, | (%, |
| Run | Buffer pH | resin) | Cycle | Time (h) | (%, predicted) | (%, actual) | predicted) | actual) |
| 1 | 4.10 | 44 | 6 | 1.68 | 76.3 | 80.4 | 6.41 | 6.20 |
| 2 | 4.25 | 44 | 57 | 15.98 | 71.7 | 78.7 | 6.43 | 5.97 |
| 3 | 4.35 | 44 | 110 | 30.84 | 80.4 | 83.2 | 6.24 | 6.22 |

The bispecific yield model consistently predicted low across the evaluation range but were within 7% of actual. Column loading was not a significant factor in the prediction of binding impurity. The binding impurity model predictions were higher than the actual, but within 0.5%.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A method of purifying a heterodimeric protein, comprising:
(a) performing a series of chromatographic cycles, wherein each cycle comprises:
(i) introducing a mixture of a heterodimeric protein and impurities to an affinity matrix containing a protein-binding ligand, wherein the heterodimeric protein comprises first and second polypeptides with differing affinity for the protein-binding ligand, and wherein at least one impurity binds the protein-binding ligand and at least one impurity does not bind the protein-binding ligand;
(ii) washing the affinity matrix with a first wash buffer at a first pH of from 5 to 9 to remove non-binding impurities;
(iii) eluting the heterodimeric protein from the affinity matrix in a first elution buffer at a second pH; and
(iv) washing the affinity matrix with a second wash buffer at a third pH of less than 4 to remove binding impurities;
wherein the second pH is at a preliminary pH during a preliminary series of cycles within the series of chromatographic cycles, and the second pH is raised to a subsequent pH higher than the preliminary pH during a subsequent series of cycles within the series of chromatographic cycles, wherein the preliminary pH and the subsequent pH are within a range of from 4.0 to 5.2; and
(b) collecting the heterodimeric protein from the affinity matrix in an eluate.

2. The method of claim 1, wherein the preliminary series of cycles consists of 20 cycles, 30 cycles, 40 cycles, 50 cycles, 60 cycles, 70 cycles, or 80 cycles.

3-5. (canceled)

6. The method of claim 2, wherein the subsequent series of cycles consists of at least 20, at least 50, at least 60, at least 70, or at least 80 cycles.

7. (canceled)

8. The method of claim 1, wherein the preliminary pH is from 4.0 to 4.2, or the preliminary pH is 4.1±0.05.

9. (canceled)

10. The method of claim 1, wherein the subsequent pH is from 4.3 to 4.7, or the subsequent pH is 4.5±0.05.

11. (canceled)

12. A method of purifying a heterodimeric protein, comprising:
(a) performing a series of chromatographic cycles, wherein each cycle comprises:
(i) introducing a mixture of a heterodimeric protein and impurities to an affinity matrix containing a protein-binding ligand, wherein the heterodimeric protein comprises first and second polypeptides with differing affinity for the protein-binding ligand, and wherein at least one impurity binds the protein-binding ligand and at least one impurity does not bind the protein-binding ligand;
(ii) washing the affinity matrix with a first wash buffer at a first pH of from 5 to 9 to remove non-binding impurities;
(iii) eluting the heterodimeric protein from the affinity matrix in a first elution buffer at a second pH; and
(iv) washing the affinity matrix with a second wash buffer at a third pH of less than 4 to remove binding impurities;
(b) measuring a level of binding impurity in an eluate containing the heterodimeric protein following any one or more of the cycles within the series of chromatographic cycles, and comparing the measured level of binding impurity to a reference level of binding impurity, wherein if the measured level of binding impurity exceeds the reference level of binding impurity, then increasing the second pH in a subsequent cycle within the series of chromatographic cycles, wherein the second pH is within a range of from 4.0 to 5.2 during each cycle or subsequent cycle within the series of chromatographic cycles; and
(c) collecting the heterodimeric protein from the affinity matrix in the eluate.

13. The method of claim 12, wherein the reference level of binding impurity is from 2% to 10%, or from 3% to 7%, or the reference level of binding impurity is 5%±0.5%.

14-15. (canceled)

16. The method of claim 12, wherein the level of binding impurity in the eluate is measured: following each cycle within the series of chromatographic cycles; following every fifth cycle in the series of chromatographic cycles; following every tenth cycle or every twentieth cycle in the series of chromatographic cycles; or following a fortieth cycle or a fiftieth cycle in the series of chromatographic cycles.

17-19. (canceled)

20. The method of claim 12, wherein the level of binding impurity in the eluate is measured in a combined eluate pool collected from a series of cycles.

21. The method of claim 12, wherein the second pH is increased to a range of from 4.3 to 4.7 from a range of from 4.0 to 4.2 if the measured level of binding impurity exceeds the reference level of binding impurity, or the second pH is increased to 4.5±0.05 from 4.1±0.05 if the measured level of binding impurity exceeds the reference level of binding impurity.

22-42. (canceled)

43. The method of claim 1, wherein the impurities comprise homodimeric species of the first and second polypeptides.

44. The method of claim 1, wherein the protein-binding ligand is Protein A, and the affinity matrix comprises the Protein A ligand affixed to a substrate, or the Protein A ligand is an engineered Protein A comprising a Z-domain tetramer, an engineered Protein A comprising a Y-domain tetramer, or an engineered Protein A that lacks D and E domains.

45. (canceled)

46. The method of claim 44, wherein
(a) the substrate is a particle and the affinity matrix comprises a multiplicity of the particles comprising a mean diameter of from 25 μm to 100 μm, from 40 μm to 60 um, from 45 μm to 55 μm, or about 50 μm;
(b) the substrate comprises any one or more of agarose, poly(styrene divinylbenzene), polymethacrylate, cellulose, controlled pore glass, and spherical silica; or
(c) the substrate is a particle and the affinity matrix comprises a multiplicity of the particles comprising pores having a mean diameter of about 1100 Å.

47-51. (canceled)

52. The method of claim 1, wherein the elution buffer comprises a salt at a concentration of at least 250 mM, or at a concentration of greater than 300 mM, or at a concentration of greater than 400 mM, or at a concentration of about 500 mM.

53-54. (canceled)

55. The method of claim 52, wherein the salt is selected from a salt containing (i) Cl⁻, Br⁻, I⁻, NO₃⁻, N(CH₃)₄⁺, NH₄⁺, Cs⁺, Rb⁺, K⁺, Na⁺, H⁺, Ca²⁺, Mg²⁺, Al³⁺; (ii) combinations of Na⁺, H⁺, Ca²⁺, Mg²⁺ or Al³⁺ with Cl⁻, Br⁻, I⁻, NO₃⁻, or ClO⁻, or (iii) CaCl₂, MgCl₂ or NaCl.

56. The method of claim 1, wherein the first polypeptide comprises a CH3 domain that is capable of binding to the protein-binding ligand and the second polypeptide comprises a CH3 domain that is not capable of binding to the protein-binding ligand.

57. The method of claim 44, wherein the first polypeptide comprises a CH3 domain that is capable of binding to Protein A and the second polypeptide comprises a CH3 domain that is not capable of binding to Protein A, or wherein the second polypeptide comprises a H435R modification and a Y436F modification (EU numbering) in the CH3 domain.

58. (canceled)

59. The method of claim 10, wherein the first pH is from 6 to 8, or wherein the third pH is from 2.8 to 3.5.

60. (canceled)

61. The method of claim 1, wherein the heterodimeric protein is an antibody, the heterodimeric protein is a bispecific antigen-binding protein, or the heterodimeric protein is a bispecific antibody.

62-63. (canceled)

64. The method of claim 1, wherein at least 85% of the heterodimeric protein is recovered in the eluate in each cycle within the series of chromatographic cycles, or at least 87% of the heterodimeric protein is recovered in the eluate in each cycle within the series of chromatographic cycles, or at least 89% of the heterodimeric protein is recovered in the eluate in each cycle within the series of chromatographic cycles.

65-66. (canceled)

67. The method of claim 1, wherein the series of chromatographic cycles comprises 100 or more cycles.

68. The method of claim 1, wherein the affinity matrix is contacted with a basic solution having a pH of at least 11 following every cycle, following every three cycles, following every five cycles, or following every seven cycles.

69-71. (canceled)

72. The method of claim 68, wherein the pH of the basic solution is at least 12, wherein the basic solution comprises a base at a concentration of from 0.1 N to 0.5 N, wherein the basic solution comprises a base at a concentration of from 0.1 N to 0.3 N, or wherein the basic solution comprises NaOH.

73-75. (canceled)

76. The method of claim 1, wherein each cycle further comprises (v) cleaning the affinity matrix by contacting the affinity matrix with a basic solution having a pH of at least 11.

77. The method of claim 76, wherein the pH of the basic solution is at least 12, wherein the basic solution comprises a base at a concentration of from 0.1 N to 0.5 N, wherein the basic solution comprises a base at a concentration of from 0.1 N to 0.3 N, or wherein the basic solution comprises NaOH.

78-80. (canceled)

81. The method of claim 76, wherein at least 75%, 78%, or 80% of the heterodimeric protein is recovered in the eluate in each cycle within the series of chromatographic cycles, and the binding impurities do not exceed

82-83. (canceled)
